(19) United States
(12) Patent Application Publication
(10) Pub. No.: US 2026/0188498 A1
(43) Pub. Date: Jul. 2, 2026

(54) SYSTEMS AND METHODS FOR RAPID IDENTIFICATION OF FUNGAL SPECIES USING COUNTER-PROPAGATING GAUSSIAN BEAM RAMAN SPECTROSCOPY AND DEEP LEARNING

(71) Applicant: Wayne State University, Detroit, MI (US)

(72) Inventors: Satya Kiran Koya, Troy, MI (US); Michelle Brusatori, Sterling Heights, MI (US); Gregory William Auner, Livonia, MI (US)

(73) Assignee: Wayne State University, Detroit, MI (US)

(21) Appl. No.: 19/437,088

(22) Filed: Dec. 30, 2025

Related U.S. Application Data
(60) Provisional application No. 63/739,820, filed on Dec. 30, 2024.

(51) Int. Cl. G16H 50/20; G01N 21/65; G16B 40/10; G16B 40/20; G16H 15/00; G16H 20/10

(52) U.S. Cl. CPC G16H 50/20; G01N 21/65; G16B 40/10; G16B 40/20; G16H 15/00; G16H 20/10; G01N 2021/651

(57) ABSTRACT

Systems and methods for identification of fungal pathogens, including Candida auris and other Candida species, using Raman spectroscopy integrated with machine learning are described. The systems and methods can utilize a counter-propagating Gaussian beam Raman spectroscopy (CPGB-RS) system in combination with a Convolutional Neural Network (CNN) that processes the Raman spectral data to identify and classify fungal species with high accuracy, sensitivity, and specificity.

### CROSS-REFERENCE TO RELATED APPLICATION

This application claims priority to and the benefit of the earlier filing date of U.S. Provisional Patent Application No. 63/739,820 filed on Dec. 30, 2024, which is incorporated herein by reference in its entirety as if fully set forth herein.

### FIELD OF THE DISCLOSURE

The present disclosure provides systems and methods for identification of fungal pathogens using Raman spectroscopy integrated with deep learning.

### BACKGROUND OF THE DISCLOSURE

Fungal pathogens cause numerous health concerns. Examples of fungal pathogens include Candida, Coccidioides, Cryptococcus, Histoplasma, Leishmania, Plasmodium, Protozoa, Schistosomae, Tinea, Toxoplasma, and Trypanosoma cruzi.

As one example, Candida auris, is a multidrug-resistant yeast classified as an “urgent threat” by the U.S. Centers for Disease Control and Prevention. The accurate identification of Candida auris is critical for mitigating healthcare-associated outbreaks. Traditional diagnostic methods, such as culture-based techniques, biochemical assays, and MALDI-TOF MS, have limitations, including extended turnaround times, misidentifications, and prohibitive costs. Polymerase Chain Reaction (PCR) methods, though accurate, are labor-intensive and expensive.

Raman spectroscopy offers a promising alternative due to its reagentless, non-destructive nature and ability to provide detailed molecular fingerprints. However, conventional Raman systems suffer from limitations, such as sensitivity challenges in detecting low-concentration samples.

### SUMMARY OF THE DISCLOSURE

The current disclosure provides systems and methods for identification of fungal pathogens, including Candida auris and other Candida species, using Raman spectroscopy integrated with deep learning. In particular embodiments, the systems and methods utilize a counter-propagating Gaussian beam Raman spectroscopy (CPGB-RS) system in combination with a Convolutional Neural Network (CNN) that processes the Raman spectral data to identify and classify Candida species with high accuracy, sensitivity, and specificity.

Exemplary beneficial aspects of the disclosed systems and methods include one or more of:
(1) Rapid and Reagentless Diagnosis: The systems can provide diagnostic results within two minutes, requiring minimal sample preparation and enabling the detection of low-concentration samples;
(2) Clinical Utility: The systems can be used in diverse healthcare settings, including resource-limited environments, offering cost-effective and efficient alternatives to conventional diagnostic modalities; and
(3) Phenotypic Resistance Detection: The systems can determine antifungal resistance or susceptibility in fungal species by analyzing changes in spectral biomarkers when exposed to antifungal agents, providing results, for example, within 30 minutes.

Thus, the disclosed systems and methods are applicable in clinical microbiology laboratories, hospitals, and point-of-care settings. They provide rapid, accurate, and cost-effective solutions for identifying multidrug-resistant fungal pathogens, significantly improving patient outcomes and infection control efforts. Furthermore, the ability to evaluate antimicrobial resistance directly from samples positions the disclosed system as critical tools in combating global antifungal resistance challenges.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. Applicant considers the color versions of the drawings as part of the original submission and reserve the right to present color images of the drawings in later proceedings

FIG. 1. Schematic of the optical element including a counter-propagating Gaussian beam (CPFB) Raman spectrometer.

FIG. 2. Confusion matrix for the test data set. The normalized confusion matrix summarizing the accuracy of results was based on the evaluation of 150 samples and 1495 spectra of the held-out test data. Each entry is shown as the number of spectra and a percentage calculated from each column's total number of entries is shown in parentheses. The vertical axis shows the ground truth classification, and the horizontal axis shows the classification predicted by the CNN model. The legend axis represents the number of spectra in each category.

FIG. 3. Mean Raman spectra and feature importance for Candida species. Mean Raman spectra for each Candida species (C. auris in solid black, C. albicans in solid red, C. glabrata in solid blue, and C. tropicalis in solid magenta), and their standard deviation, shown in respective transparent colors, are shown in the upper panel of the figure. The bottom panel shows feature importance calculated using permutation importance analysis. Feature importance reveals how variables contribute to model predictions. Positive values indicate performance enhancement, while negative values suggest complex, non-linear relationships that require careful investigation and context-aware interpretation. The vertical axis for the upper panel shows peak intensity in arbitrary units and the bottom panel shows the importance of each peak (positive or negative) in arbitrary units with an axis break (≈). The horizontal axis common for both panels shows Raman shift frequencies.

FIG. 4. Investigation of Cytochrome c in the supernatant. The mean Raman spectra of C. auris cell suspension at OD of 2.0 and supernatant were plotted. The horizontal axis shows the Raman shift frequencies, and the vertical axis shows peak intensities in arbitrary units. The arrows indicate cytochrome c in the cell suspension.

FIG. 5. Differences in the Raman peaks in four Candida species. Bar graph showing mean Raman spectra and its standard error (error bars) for Raman peaks belonging to cellular energy states (750, 1128 and 1337 cm⁻¹), cell wall composition (893, 974, 750, and 1054 cm⁻¹), and cell membrane constituents (597, 620, 1602 and 1666 cm⁻¹) were plotted for C. auris, C. albicans, C. glabrata, and C. tropicalis. The horizontal axis shows Raman shift, and the vertical axis shows peak intensity in arbitrary units.

### DETAILED DESCRIPTION

Fungal pathogens cause numerous health concerns. Examples of fungal pathogens include Candida, Coccidioides, Cryptococcus, Histoplasma, Leishmania, Plasmodium, Protozoa, Schistosomae, Tinea, Toxoplasma, and Trypanosoma cruzi.

As one example, Candida auris, is a multidrug-resistant yeast classified as an “urgent threat” by the U.S. Centers for Disease Control and Prevention. The accurate identification of Candida auris is critical for mitigating healthcare-associated outbreaks. Traditional diagnostic methods, such as culture-based techniques, biochemical assays, and MALDI-TOF MS, have limitations, including extended turnaround times, misidentifications, and prohibitive costs. Polymerase Chain Reaction (PCR) methods, though accurate, are labor-intensive and expensive.

Raman spectroscopy offers a promising alternative due to its reagentless, non-destructive nature and ability to provide detailed molecular fingerprints. However, conventional Raman systems suffer from limitations, such as sensitivity challenges in detecting low-concentration samples. The current disclosure utilizes a specialized optical arrangement to achieve high-sensitivity Raman measurements at low target concentrations without the need for chemical enhancement agents.

The current disclosure provides systems and methods for identification of fungal pathogens, including Candida auris and other Candida species, using Raman spectroscopy integrated with deep learning. In particular embodiments, the systems and methods utilize a counter-propagating Gaussian beam Raman spectroscopy (CPGB-RS) system in combination with machine learning (ML). In particular embodiments, the systems and methods utilize a convolutional neural network (CNN).

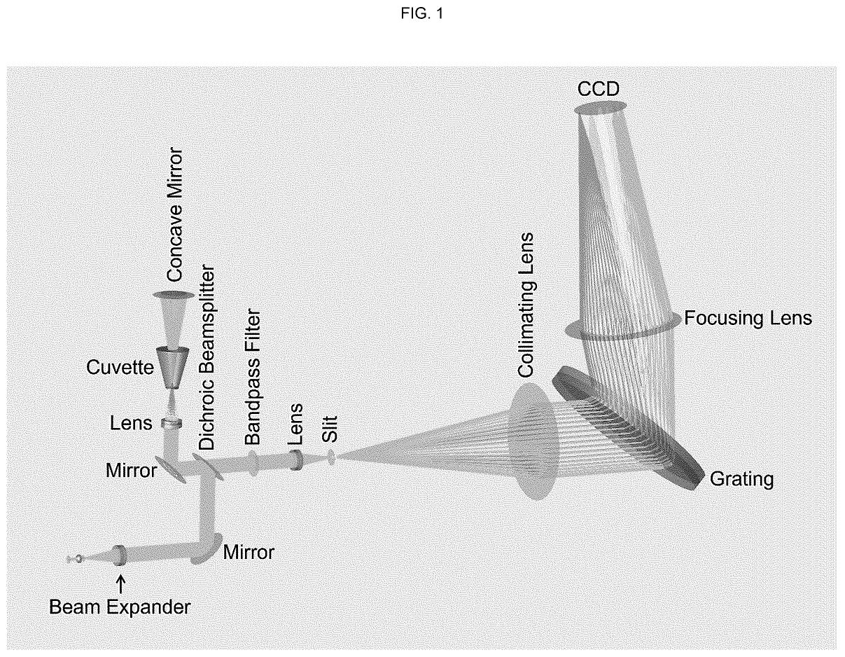

In particular embodiments, a CPGB-RS system refers to a portable Raman spectrometer equipped with a 532 nm laser for excitation. In particular embodiments, the system includes a cuvette design. The system's cuvette design concentrates particles at the laser's focal point, enhancing signal collection. Raman-scattered light is filtered and directed through a high-resolution spectrometer, which separates wavelengths onto a cooled CCD detector for data acquisition (FIG. 1).

ML refers to the use of a computing device to learn patterns in training data. The process of learning these patterns may be referred to as “training.” In particular cases, one or more computing devices may perform machine learning by executing a machine learning model.

ML models refer to data encoding instructions that, when executed by at least one computing device, cause the at least one computing device to learn patterns in training data by optimizing one or more metrics, values, or other types of parameters. After training, an ML model, when executed by at least one computing device, causes the at least one computing device to utilize the optimized parameters in order to perform one or more tasks.

A CNN refers to a type of ML model configured to identify features in input data by performing a series of convolutions or cross-correlations on the input data with multiple filters (also referred to as “kernels”). In various cases, the input data for a CNN is in the form of an image. In various cases, a CNN is defined according to multiple layers (also referred to as “blocks”), which may be arranged in parallel and/or series, wherein each layer is defined according to a filter. Each layer, for instance, corresponds to a convolution and/or cross-correlation operation between the input data for the layer and the filter that defines the layer. The output of each layer is provided as input data for a subsequent layer or is output from the CNN. In some cases, individual layers further define pooling and/or normalization functions.

The term image refers to a 2D or 3D array of data indicative of an array of pixels or voxels. For instance, colors, values, saturations, intensities, spectra, or a combination thereof, of the pixels or voxels are indicative of the data. A “digital image,” for instance, refers to digital data indicative of an image.

The terms “transform,” “data transform,” and their equivalents, refer to a process for converting a dataset from one domain to another domain. In various cases, transforms are reversible. Data that has been generated as a result of a transform may be referred to as “transformed data.”

The term “domain,” and its equivalents, refer to a set of possible inputs and/or a set of independent variables of a function or dataset. In some cases, if a dataset includes ordered pairs of first and second elements, wherein the second elements are respectively dependent on the first elements, then the domain of that dataset includes the first elements.

The term “filter,” and its equivalents, refer to a system that performs one or more mathematical operations on a signal or dataset in order to reduce or enhance aspects of the signal or dataset.

When using a CPGB-RS system combined with ML to identify a fungal species according to the current disclosure, the following steps and considerations can be taken into account:

Sample Preparation. Biological samples can be prepared by washing fungal cells with sterile saline and suspending them at a defined optical density (e.g., OD at 600 nm). Samples can be placed in a custom cuvette for spectral analysis. The cuvette may be a tapered-walled cuvette. The cuvette has a unique design to concentrate particles at a focal point (e.g., 8-μm focal point). Biological samples, including fungal cells, can be obtained from a subject undergoing a diagnostic procedure.

Data Acquisition and Preprocessing. The Raman spectra can be recorded over a broad range of 285-1925 cm⁻¹. A 532 nm laser source and a high-resolution spectrometer (e.g., 2650 L/mm grating) can be used. Preprocessing steps can include baseline subtraction, spectral range optimization (540-1740 cm⁻¹), and normalization to improve signal-to-noise ratios. An open morphology weighted penalized least square method can be used for baseline subtraction in order to isolate the high-fidelity molecular fingerprint from background interferences. Spectral range optimization is focused on the 540-1740 cm⁻¹ spectral window, which contains high-density information regarding cellular proteins, lipids, and carbohydrates. Signal-to-noise ratios are further improved through unit vector normalization and a third-order smoothing operation. Outlier spectra can be removed to ensure data integrity.

Deep Learning Algorithm. The system can employ a CNN architecture for spectral analysis. The algorithm can extract features from the Raman spectra to differentiate fungal species (e.g., Candida auris) from other species based on unique molecular fingerprints. Specifically, a one-dimensional (1D) convolutional neural network (1D-CNN) can be used for feature extraction from vibrational spectral. The model includes at least three successive convolutional layers (Conv1D) with filter sizes of 32, 128, and 512, respectively. This allows the system to identify both simple peaks and complex nonlinear interactions between spectral features. Training and testing datasets can be used to optimize model performance, achieving, for example, an accuracy of 96%, sensitivity of 96%, and specificity of 99%.

Key Raman Features. The differentiation between fungal (e.g., Candida) species can be based on variations in: cell wall composition, cell membrane components, and/or cellular energy states. Cell wall composition refers to unique spectral features arising from β-glucan, chitin, and mannoprotein. Cell membrane components refer to ergosterol-specific Raman peaks. Cellular energy states refer to Raman features indicative of mitochondrial cytochromes b and c.

Advanced Spectral Analysis Techniques. To further enhance the system's capabilities, additional spectral processing techniques can be employed. Examples of these additional spectral processing techniques include the following.

High-Resolution Feature Extraction. The system leverages a multi-layer CNN structure to identify hierarchical spectral patterns that distinguish closely related species;

Adaptive Learning Algorithms. Periodic updates to the deep learning model ensure adaptability to new fungal strains or species with minimal manual intervention; and/or

Real-Time Anomaly Detection. A secondary diagnostic module flags unusual spectra indicative of rare or emerging pathogens for further analysis.

Integrated Antifungal Resistance Monitoring. The system can evaluate the effectiveness of antifungal agents by analyzing spectral changes over time. This functionality aids in monitoring resistance development and optimizing therapeutic strategies. Additionally, resistance determination is achieved by exposing pathogens to antibiotics and monitoring spectral changes indicative of susceptibility or resistance.

System Operation. The CPGB-RS system automates spectral acquisition, preprocessing, and analysis. Diagnostic results are generated and displayed within two minutes, facilitating real-time clinical decision-making. Advanced features such as batch analysis and remote diagnostics further enhance usability. The system supports cloud-based updates to integrate new spectral databases for emerging pathogens and antibiotic resistance patterns.

In particular embodiments, the system and methods described herein are configured to generate a report based on the fungal species identified by the deep learning model. The report provides a comprehensive classification analysis, which may be generated using a sci-kit-learn's classification_report function. The report is a multi-dimensional assessment of the model's confidence and reliability.

Beyond identification, in particular embodiments, the systems and methods are configured to provide a recommended therapy based on the identified fungal species and its associated resistance profile. In particular embodiments, the recommended therapy includes a dosage of one or more therapeutic agents predicted to treat a condition of a subject associated with the identified fungal species.

In particular embodiments, the system is configured to perform adaptive learning for new species identification. The training set to train the machine learning model may include new species. In particular embodiments, the system includes an adaptive learning module that utilizes a Raman spectral database with updated and verified spectral fingerprints. The machine learning model may undergo training with the weights of the existing filters to recognize new markers (e.g., variations in cell wall chitin or membrane ergosterol).

In particular embodiments, the system includes a real-time anomaly detection module configured to detect and identify unusual spectra in real time. This module may serve as a secondary layer to evaluate the input Raman spectrum before or after the classification. In particular embodiments, the system and methods described herein are configured to utilize spectral biomarkers indicative of antimicrobial resistance or susceptibility to determine minimum inhibitory concentrations. The system evaluates spectral biomarkers indicative of antimicrobial resistance or susceptibility by monitoring changes in the molecular fingerprint of a fungal sample following exposure to varying concentrations of one or more therapeutic agents.

In particular embodiments, the system provides a rapid assessment of MIC and susceptibility profiles, generating results within a significantly compressed timeframe, such as within 30 minutes.

Exemplary advantages of disclosed systems and methods include one or more of: speed (e.g., diagnostic results within two minutes and resistance profiles within 30 minutes; accuracy (e.g., high sensitivity (96%) and specificity (99%)); portability (e.g., compact design suitable for diverse clinical settings); cost-effectiveness (e.g., reagentless operation reduces per-test costs); scalability (e.g., potential for integration with broader diagnostic workflows and expansion to other pathogens); versatility (e.g., capable of monitoring antifungal resistance and identifying emerging pathogens); and cloud-enabled updates: (e.g., rapid integration of new pathogen and resistance data).

Particular and preferred embodiments disclosed herein utilize a wavelength of 532 nm. Other embodiments utilizing other wavelengths can also be explored. For example, in some embodiments, a Raman spectrometer includes a multimode diode laser having a wavelength from 300 nm to 1200 nm, from 350 nm to 1100 nm, from 400 nm to 1100 nm, from 400 nm to 1064 nm, from 450 nm to 1064 nm, from 500 nm to 1064 nm, from 550 nm to 1064 nm, from 600 nm to 1064 nm, from 650 nm to 1064 nm, from 700 nm to 1064 nm, from 450 nm to 1100 nm, or from 500 nm to 1100 nm. In some embodiments, a light source is a narrow bandwidth laser with a wavelength of 800 nm, 785 nm, 750 nm, 725 nm, 700 nm, 675 nm, 650 nm, 625 nm, 600 nm, 575 nm, 550 nm, 532 nm, 525 nm, or 500 nm.

Embodiments disclosed herein can utilize a computer architecture capable of executing program components for implementing the functionality described herein. The computer architecture can include a conventional computer, workstation, desktop computer, laptop, tablet, network appliance, e-reader, smartphone, or other computing device, and can be utilized to execute any of the processes described herein.

The computer includes a baseboard or “motherboard,” which is a printed circuit board to which a multitude of components or devices can be connected by way of a system bus or other electrical communication paths. In one illustrative configuration, one or more processing units, such as (“CPUs”), GPUs, TPUs, ASICs, FPGAs, or the like, and/or threads, kernels, cores, and/or the like thereof, that may operate in conjunction with a chipset. The CPUs can be standard programmable processors that perform arithmetic and logical operations necessary for the operation of the computer.

The CPUs perform operations by transitioning from one discrete, physical state to the next through the manipulation of switching elements that differentiate between and change these states. Switching elements generally include electronic circuits that maintain one of two binary states, such as flip-flops, and electronic circuits that provide an output state based on the logical combination of the states of one or more other switching elements, such as logic gates. These basic switching elements can be combined to create more complex logic circuits, including registers, adders-subtractors, arithmetic logic units, floating-point units, and the like.

The chipset provides an interface between the CPUs and the remainder of the components and devices on the baseboard. The chipset can provide an interface to a random-access memory (RAM) or any other suitable form of memory, used as the main memory in the computer. The chipset can further provide an interface to a computer-readable storage medium such as a read-only memory (ROM) or non-volatile RAM (NVRAM) for storing basic routines that help to startup the computer and to transfer information between the various components and devices. The ROM or NVRAM can also store other software components necessary for the operation of the computer in accordance with the configurations described herein.

The computer can operate in a networked environment using logical connections to remote computing devices and computer systems through a network. The chipset can include functionality for providing network connectivity through a network interface controller (NIC), such as a gigabit Ethernet adapter. The NIC is capable of connecting the computer to other computing devices over the network. It should be appreciated that multiple NICs can be present in the computer, connecting the computer to other types of networks and remote computer systems. In some instances, the NICs may include at least on ingress port and/or at least one egress port.

The computer can include an input/output (I/O), such as a controller sufficient to transmit processor-executable instructions to or receive processor-executable instructions from a device. For example, the I/O controller include or interface with one or more user interface devices (e.g., a display, speaker, a keyboard, a mouse, a trackpad, a touchscreen), one or more servers, laboratory equipment (e.g., spectrometers), and/or the like. Interfacing with any of these devices may additionally or alternatively be executed by the network interface controller.

The computer can be connected to a storage device that provides non-volatile storage for the computer. The storage device can store an operating system, programs, and data. The storage device can be connected to the computer through a storage controller connected to the chipset. The storage device can consist of one or more physical storage units. The storage controller can interface with the physical storage units through a serial attached small computer system interface (SCSI) (SAS) interface, a serial advanced technology attachment (SATA) interface, a fiber channel (FC) interface, or other type of interface for physically connecting and transferring data between computers and physical storage units.

The computer can store data on the storage device by transforming the physical state of the physical storage units to reflect the information being stored. The specific transformation of physical state can depend on various factors, in different embodiments of this description. Examples of such factors can include the technology used to implement the physical storage units, whether the storage device is characterized as primary or secondary storage, and the like.

For example, the computer can store information to the storage device by issuing instructions through the storage controller to alter the magnetic characteristics of a particular location within a magnetic disk drive unit, the reflective or refractive characteristics of a particular location in an optical storage unit, or the electrical characteristics of a particular capacitor, transistor, or other discrete component in a solid-state storage unit. Other transformations of physical media are possible without departing from the scope and spirit of the present description, with the foregoing examples provided only to facilitate this description. The computer can further read information from the storage device by detecting the physical states or characteristics of one or more particular locations within the physical storage units.

In addition to the storage device described above, the computer can have access to other computer-readable storage media to store and retrieve information, such as program modules, data structures, or other data. It should be appreciated by those skilled in the art that computer-readable storage media is any available media that provides for the non-transitory storage of data and that can be accessed by the computer. In some examples, the operations performed by any network node described herein may be supported by one or more devices similar to computer. Stated otherwise, some or all of the operations performed by a network node may be performed by one or more computer devices operating in a cloud-based arrangement.

By way of example, computer-readable storage media can include volatile and non-volatile, removable and non-removable media implemented in any method or technology. Computer-readable storage media include RAM, ROM, erasable programmable ROM (“EPROM”), electrically-erasable programmable ROM (“EEPROM”), flash memory or other solid-state memory technology, compact disc ROM (“CD-ROM”), digital versatile disk (“DVD”), high definition DVD (“HD-DVD”), BLU-RAY, or other optical storage, magnetic cassettes, magnetic tape, magnetic disk storage or other magnetic storage devices, or any other medium that can be used to store the desired information in a non-transitory fashion.

As mentioned briefly above, the storage device can store an operating system utilized to control the operation of the computer. According to one embodiment, the operating system includes the LINUX® operating system. According to another embodiment, the operating system includes the WINDOWS SERVER® operating system from MICROSOFT Corporation of Redmond, Washington. According to further embodiments, the operating system can include the UNIX® operating system or one of its variants. It should be appreciated that other operating systems can also be utilized. The storage device can store other system or application programs and data utilized by the computer.

In one embodiment, the storage device or other computer-readable storage media includes one or more programs. The programs, for example, include computer-executable instructions which, when loaded into the computer, transform the computer from a general-purpose computing system into a special-purpose computer capable of implementing the embodiments described herein. These computer-executable instructions transform the computer by specifying how the CPUs transition between states, as described above. According to one embodiment, the computer has access to computer-readable storage media storing computer-executable instructions which, when executed by the computer, perform the various processes described herein. The computer can also include computer-readable storage media having instructions stored thereupon for performing any of the other computer-implemented operations described herein. The program(s), for example, include one or more processes. The process(es) may include instructions that, when executed by the CPU(s), cause the computer and/or the CPU(s) to perform one or more operations.

The computer can also include one or more input/output controllers for receiving and processing input from a number of input devices, such as a keyboard, a mouse, a touchpad, a touch screen, an electronic stylus, or other type of input device. Similarly, an input/output controller can provide output to a display, such as a computer monitor, a flat-panel display, a digital projector, a printer, or other type of output device. It will be appreciated that the computer might not include all of the described components, can include other components that are not explicitly described, or might utilize an architecture completely different than that as described.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

The Experimental Example below is included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Experimental Example. Rapid Identification of Candida auris by Raman Spectroscopy Combined with Deep Learning

Abstract. Candida auris is a highly pathogenic yeast first identified in Japan in 2009 and has become a significant global health concern. This yeast often resists multiple drugs, which makes it challenging to identify using standard laboratory methods. Even with strict infection prevention and control measures in healthcare settings, it can still lead to outbreaks within healthcare facilities. Most Candida species, including C. auris, are pathogenic, but distinguishing C. auris from other pathogenic Candida species is crucial due to its unique resistance profiles and outbreak potential. This example has introduced a practical, portable, and reagentless platform called counter-propagating Gaussian beam Raman spectroscopy (CPGB-RS) integrated with deep learning spectral analysis. This platform allows for the rapid and accurate identification and differentiation of C. auris from the most common pathogenic Candida species, such as albicans, glabrata, and tropicalis. It has a sensitivity of 96% and a specificity of 99% when analyzing cultures. The differentiation between species is based on unique variations in the Raman spectra, which are influenced by differences in cell wall composition (β-glucan, chitin, and mannoprotein), cell membrane components (ergosterol), and cellular energy states (mitochondrial cytochromes b and c). The platform enables automated molecular screening with a software-generated diagnostic result in just 2 minutes, making it highly practical for clinical applications. Additionally, this technology has the potential to assess the effectiveness of antifungal agents and thus could significantly impact patient outcomes. At least some of the work described herein was published in Koya, et al. (2025). Rapid Identification of Candida auris by Raman Spectroscopy Combined With Deep Learning. Journal of Raman Spectroscopy, 56 (3), 218-227.

Introduction. Candida auris is an escalating global health threat, capable of inducing severe infections with high mortality rates. The Centers for Disease Control (CDC) rates this yeast as an “urgent threat” due to its multidrug resistance and higher incidence of nosocomial infections. C. auris persists on environmental surfaces despite increased infection prevention and control measures. Accurate identification of C. auris is a significant challenge through conventional microbiological growth tests and biochemical methods. These often misidentify the species or provide only genus-level identification. Diagnostic modalities based on cellular morphology, such as culturing on chromogenic media, do not differentiate C. auris because it lacks a distinctive colony color. On the novel chromogenic media, CHROMagar Candida Plus (CHROMagar, France), C. auris forms pale cream colonies with a distinctive blue halo. However, other rare nosocomial Candida species, such as C. vulturna and C. pseudohaemulonii, also form pale cream colonies with a distinctive blue halo. Diagnostic modalities employing phenotypic and biochemical methods based on growth characteristics either in non-automated or automated forms (Vitek 2 YST, API 20C, API ID 32C, BD Phoenix, MicroScan, and RapID Yeast Plus) frequently misidentify with C. auris with closely related but uncommon yeasts such as C. haemulonii, C. pseudohaemulonii, and C. duobushaemulonii rendering them unreliable. Due to the shortcomings of these methods, matrix-assisted laser desorption/ionization time-of-flight mass spectrometry (MALDI-TOF MS) and polymerase chain reaction (PCR) diagnostic techniques are used to detect C. auris. MALDI-TOF MS demonstrates robust proficiency in directly identifying all isolates of C. auris from culture plates or blood cultures. However, further subculturing may be necessary in mixed-species cultures. Although MALDI offers a high detection accuracy, its high upfront cost can be prohibitive for smaller community hospitals or diagnostic facilities and requires sample pretreatment prior to spectral acquisition. Methods leveraging the distinct features of glycosylphosphatidylinositol (GPI)-modified protein-encoding genes of C. auris, such as conventional polymerase chain reaction (PCR) that target the D1-D2 region of the 28s ribosomal DNA (rDNA) or the internal transcribed spacer (ITS) region of rDNA using multiplex PCR or genome sequencing, have proven effective in the identification and differentiating C. auris. Nevertheless, these techniques are labor intensive, expensive, and require complex sample preparation.

Raman spectroscopy (RS) is an emerging modality for yeast identification. Unlike MALDI-TOF MS and PCR, the technique is non-destructive, allowing further sample analysis if needed. It requires minimal sample preparation and can be used for real-time monitoring to expedite the detection of C. auris. RS is a reagentless, vibrational spectroscopic technique that provides crucial insights into the molecular structure, composition, and inter-molecular interactions, offering a spectral fingerprint by which molecules can be identified. To obtain a high-fidelity spectral fingerprint, especially among the low target concentration samples, amplifying the intensity of Raman scattered light is imperative. Surface Enhanced Raman Spectroscopy (SERS) is a dilute or trace sample analysis technique. Signal enhancement is achieved by placing the sample near/onto a metallic nanostructured surface. However, translating SERS-based methods to point-of-care can be challenging. High background and substate signals, interference from untargeted compounds, and variations in sample processing can cause irreproducible results. Unlike conventional substrate enhancement methods used in large RS microscope-based systems, the novel table-top CPGB-RS system has been specifically designed to enhance the sensitivity of RS for detecting low-concentration pathogens. This advancement leverages a counter-propagating Gaussian beam (CPGB) focused within the sample chamber to amplify the intensity of Raman scattered light. In our previous work, the CPGB-RS system with machine learning spectral analysis successfully identified and classified nine respiratory pathogens, including six viruses and one bacterium in a human nasal mucosal matrix, achieving an impressive 99% sensitivity and 93% specificity. With its ability to discern subtle variations in biomolecular fingerprints, this technology holds great promise in infectious disease management, offering a more efficient and accurate pathogen detection and classification method. The operational simplicity of the CPGB-RS system allows it to be utilized by minimally trained personnel, with an automated software-generated diagnostic read-out provided in just two minutes, rendering it highly suited for clinical applications.

The current disclosure provides a CPGB-RS system with deep learning analysis to evaluate the feasibility of accurately identifying C. auris among the most prevalent nosocomial Candida species: C. albicans, C. glabrata, and C. tropicalis. The deep learning model was trained to differentiate C. auris from the other yeast while identifying each species individually. From cultures, this method demonstrated an accuracy of 96%, sensitivity of 96%, and specificity of 99% in classifying the four Candida species, with appropriate statistical methods employed to validate these findings. This work provides for utilizing RS in C. auris identification.

Materials and Methods. Raman spectrometer system. The Raman spectrometer system used in this analysis was previously described in detail Auner et al., Biosensors and Bioelectronics: X, 2022, 12, 100230. It is a portable Raman system measuring 53 cm×33 cm×43 cm and equipped with a 532 nm laser for excitation, which is ideal for identifying pathogens. The laser beam is focused onto the sample in a specially designed cuvette to concentrate particles at the laser's focal point (FIG. 1). The scattered light from the sample is collected in a 180-degree backscatter geometry and then filtered to remove the laser contribution. The Raman signal is directed through the system's spectrometer portion, designed with a f/value of 4. The spectrometer contains a high-resolution grating (2650 l/mm) that separates light into its constituent wavelengths. The light is focused onto a 2048×70-pixel cooled CCD detector, allowing for the simultaneous detection of multiple wavelengths. The cuvette design and the particle-laser dynamics enable low-concentration measurements, especially for small particles such as viruses.

Sample Preparation. The following Candida species were obtained from the American Type Culture Collection (ATCC): C. albicans (ATCC 10231), C. auris (ATCC CDC B11903), C. glabrata (ATCC 2001), and C. tropicalis (ATCC 66029). Candida samples were prepared from Sabouraud Dextrose Emmons Agar plates. A total of 580 unique biological samples from cultures were tested for the four Candida species (Table 1).

TABLE 1
Sample number.

| Class/Yeast | Samples | Spectra | Train Spectra | Test Spectra |
|---|---|---|---|---|
| C. auris | 140 | 1401 | 1026 | 375 |
| C. albicans | 135 | 1350 | 990 | 360 |
| C. glabrata | 175 | 1755 | 1325 | 430 |
| C. tropicalis | 130 | 1294 | 964 | 330 |

A single colony was added to 10 ml of Sabouraud Dextrose broth in a 14 ml culture tube. The culture tube was placed on a shaker at 25° C. and incubated for 24 hours. Cells were washed twice using sterile saline and centrifuged at 3500 rpm for 5 min. After the final wash, the yeast pellet resuspended in saline for an optical density (OD) of 2.0±0.1 (OD at 600 nm).

Raman Spectra Acquisition. A 200 μL yeast cell suspension with an optical density (OD) of 2.0±0.1 was placed in a custom tapered-walled cuvette. The cuvette was sealed and inserted into the CPGB-RS for testing. A lens with a numerical aperture of 0.2, and a focal length of 50 mm was used to focus an 8 mm diameter laser beam to an 8-μm spot (determined using Ansys Zemax OpticStudio). For this spot, the 100-mW laser irradiation corresponded to an estimated power density of 0.4 MW/cm2, assuming a Gaussian beam profile. Ten spectra per sample were recorded using 100% laser power (100 mW at the sample) over a spectral range of 285-1925 cm⁻¹. The recording involved 24 aggregates at an integration time of 5 seconds per spectrum.

Preprocessing Raman Spectra. After obtaining the data over a spectral range of 285-1925 cm⁻¹, Raman spectra were adjusted by subtracting the baseline across a 540-1740 cm⁻¹ spectral range. This specific range was chosen because it contains the most important Raman peaks associated with cellular components such as proteins, lipids, and carbohydrates, making it the most relevant portion of the spectra for preprocessing. The regions outside this range (285-540 cm⁻¹ and 1740-1925 cm⁻¹) typically contain minimal diagnostic information and higher noise levels for fungal samples (associated with the fused silica of the cuvette window), so excluding them helps optimize the signal-to-noise ratio for subsequent analysis. This process used an open morphology weighted penalized least square method with ten successful stops and a third-order smoothing operation, which was carried out using in-house software developed in LabView (Perez-Pueyo et al., Applied Spectroscopy 2010, 64, 595). The open morphology technique identifies local minimum points and constructs a weighted vector using penalized least squares to fit the background. Following the baseline subtraction, the spectral data was normalized using a unit vector. To ensure better interpretability and greater robustness for subsequent classification using a deep learning model, exploratory data analysis was conducted to remove spectral outliers caused by oversaturated fluorescent or cosmic ray contributions.

Deep Learning Analysis. Train test split. After removing outliers and preprocessing the spectral data, all spectra were randomly divided into a training set (80% of the data) and a test set (20% of the data), as shown in Table 1. Spectra from each individual sample (consisting of 10 spectra) were assigned to either the training or test set, but not both.

Feature normalization. Feature normalization is a crucial preprocessing step in deep learning. It involves adjusting the scale of input features to address issues related to varying magnitudes (Banko & Brill, In Proceedings of the 39th annual meeting of the Association for Computational Linguistics 2001, p 26). The input features were normalized by subtracting the mean and dividing by the standard deviation calculated from the training set. This meticulous approach is important to prevent data leakage during normalization. Using statistics from the test set for normalization could compromise the model's ability to generalize to unseen data. Using statistics exclusively from the training set, the model is trained to mimic real-world scenarios, where the statistics of new, unseen data are unknown during the training phase. This enhances the model's robustness and generalization performance.

Convolutional Neural Network (CNN). The example employed a Convolutional Neural Network (CNN) architecture, a deep learning model, for feature extraction and classification tasks. The CNN model was implemented using the Keras library, a Python interface for building neural networks, with TensorFlow (open-source software library) as the backend computational engine (Chollet, et al., 2015; Abadi et al., arXiv preprint arXiv: 1603.04467 2016). The input layer of CNN accepts one-dimensional (1D) sequences of spectral data. The model consists of successive convolutional layers (Conv1D), which apply filters of increasing sizes (32, 128, and 512) to the input data. These filters employ Rectified Linear Unit (ReLU) activation functions, introducing non-linearity to capture complex patterns effectively (Agarap, arXiv preprint arXiv: 1803.08375 2018). After each convolutional layer, batch normalization is applied to improve model stability and convergence speed (Ioffe & Szegedy, In International conference on machine learning; pmlr: 2015, p 448). Max pooling operations for 1D temporal data layers (MaxPooling1D) are incorporated to down-sample the spatial dimensions while retaining essential features of the input data. These layers select the maximum value from a set of adjacent values, reducing computational complexity while preserving crucial information. The model combines the information from previous layers into a single representation by averaging across each feature map, which is essential for effective classification. The output layer uses a softmax activation function to calculate class probabilities for multiple classes. The model's hyperparameters were fine-tuned to achieve the best performance by selecting optimal values for each parameter. For the convolutional layers, 32, 128, and 512 filters with a kernel size of 5 were used for the first, second, and third layers, respectively, to effectively capture and build upon spatial hierarchies in the data. After these layers, a dense layer with 256 units integrated the extracted features for final predictions. The learning rate was set to 0.00227 to facilitate efficient learning and convergence. These hyperparameters were chosen through extensive experimentation to balance model complexity, computational efficiency, and accuracy. The CNN model architecture is summarized in Table 2, which provides information on the layer types, output shapes, and the number of parameters for each layer.

TABLE 2
Convolutional Neural Network (CNN) model architecture.

| Layer (type) | Output Shape (1D) | No. of Parameters |
|---|---|---|
| Input Layer | (1497, 1) | 0 |
| Conv1D - 1 | (1493, 32) | 192 |
| Batch Normalization | (1493, 32) | 128 |
| Max Pooling 1D | (746, 32) | 0 |
| Conv1D - 2 | (742, 128) | 20608 |
| Batch Normalization | (742, 128) | 512 |
| Max Pooling 1D | (371, 128) | 0 |
| Conv1D - 3 | (367, 512) | 328192 |
| Batch Normalization | (367, 512) | 2,048 |
| Global Average Pooling 1D | (512) | 0 |
| Dense | (256) | 131328 |
| Dense | (4) | 1028 |
| Total params | | 484036 |
| Trainable params | | 482692 |
| Non-trainable params | | 1344 |

Statistical Analysis and Prediction Performance. The CNN model's real-world performance was evaluated using the carefully prepared test dataset, to assess its classification accuracy. This test set included 1,495 spectra randomly selected from the original dataset, ensuring they were not used during the training phase. The model's predictions were generated using this dedicated test set. A confusion matrix was constructed to provide a detailed view of the model's classification accuracy across all classes by systematically comparing the model's predictions to the true labels. Furthermore, a comprehensive classification report was generated using sci-kit-learn's classification_report function. This report included key performance metrics such as precision (the ratio of correctly identified positive cases to the total predicted positive cases), recall (the proportion of correctly identified positive cases out of all actual positive cases), and F1-score (a balanced assessment of the model's performance, calculated as the harmonic mean of precision and recall). To evaluate statistical significance, several analyses were performed: overall accuracy was calculated with a 95% confidence interval using the Wilson score interval method; precision and recall for each class were computed with 95% confidence intervals using the binomial proportion confidence interval; a one-way ANOVA was conducted to compare classification accuracy across different classes; and post-hoc Tukey HSD tests were performed for pairwise comparisons between classes. All statistical analyses were conducted using Python's SciPy and statsmodels libraries, with a significance level set at α=0.05 (Pedregosa et al., The Journal of machine Learning research 2011, 12, 2825).

Feature importance. To determine the importance of each feature (represented by Raman peaks) in predicting the outcome of the CNN model, a permutation importance analysis, also known as mean decrease accuracy analysis was conducted (Breiman, Machine learning 2001, 45, 5). This analysis used the permutation importance function from the ELI5 Python package, which interfaces with the scikit-learn machine learning library (Pedregosa et al., The Journal of machine Learning research 2011, 12, 2825; Andrey Shcherbakov, 2019). The accuracy score from the Keras library (Chollet, et al., 2015) was used as the evaluation metric.

In this analysis, each feature in the test dataset was systematically permuted. This approach helps quantify the decrease in the model's accuracy score when a specific feature is randomly rearranged, effectively removing its predictive power. The resulting change in model accuracy indicates the importance of each feature. These permutation importance values were systematically quantified and visually represented. This thorough analysis helped pinpoint critical features and their impact on the CNN model's ability to differentiate, enabling better understanding and improvement of the model.

Results and discussion. Deep learning classification. This study demonstrates the effectiveness of the CPGB-RS system combined with a deep learning multiclass CNN model to accurately identify and differentiate C. auris from frequently encountered Candida species: C. albicans, C. glabrata, and C. tropicalis. The performance of the trained CNN model was rigorously evaluated using previously unseen (held-out) test data to ensure the results reflect real-world conditions.

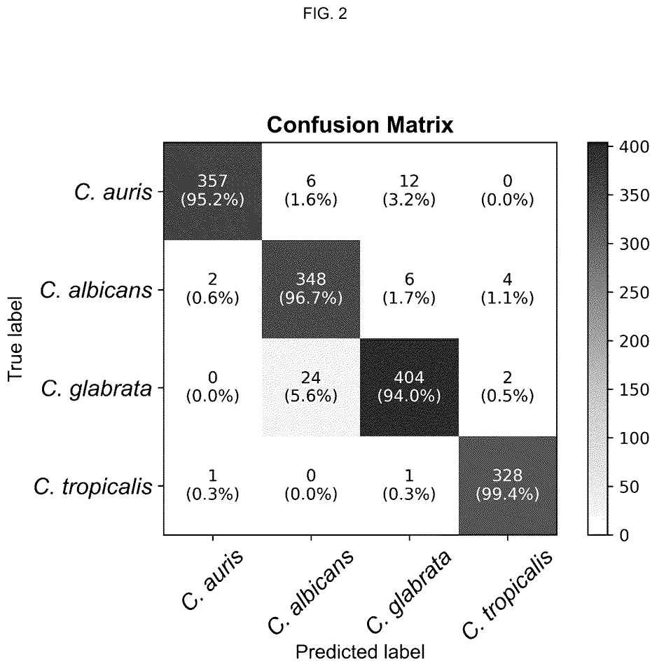

The confusion matrix (FIG. 2) visually represents the model's classification performance across all four Candida species. The model achieved a notable overall test accuracy of 96%, with a weighted sensitivity of 96% and a weighted specificity of 99%, underscoring the model's robustness. Further performance metrics, such as precision, recall, and F1-score, which balances precision and recall for each species, are summarized in Table 3.

TABLE 3
Multiclass classification report for the test data set.

| Class | Precision | Recall | f1-score | Support |
|---|---|---|---|---|
| C. auris | 0.99 | 0.95 | 0.97 | 375 |
| C. albicans | 0.92 | 0.97 | 0.94 | 360 |
| C. glabrata | 0.96 | 0.94 | 0.95 | 430 |
| C. tropicalis | 0.98 | 0.99 | 0.99 | 330 |
| Accuracy | | | 0.96 | 1495 |
| Macro avg | 0.96 | 0.96 | 0.96 | 1495 |
| Weighted avg | 0.96 | 0.96 | 0.96 | 1495 |

| | |
|---|---|
| Sensitivity (Weighted) | 0.96 |
| Specificity (Weighted) | 0.99 |

Precision measures the accuracy of predictions for each class, while recall indicates the ability of the model to identify relevant instances for each class. The f1-score is a performance metric that balances precision and recall in a single score. Support shows the number of actual spectra in each class, and accuracy shows the overall effectiveness of the model. The macro average shows average precision, recall, and f1-score across all classes. The weighted average is like the macro average but considers class support. Similarly, the weighted sensitivity and specificity, which represent the true positive and negative rates are weighted by class support.

The values for precision range from 0.92 to 0.99, recall from 0.94 to 0.99, and F-1 score from 0.94 to 0.99, further confirm the model's high level of accuracy. This result illustrates the CPGB-RS system's use for rapid and accurate identification of Candida species in clinical settings, positioning it as a valuable tool for improving diagnostic workflow and patient outcomes.

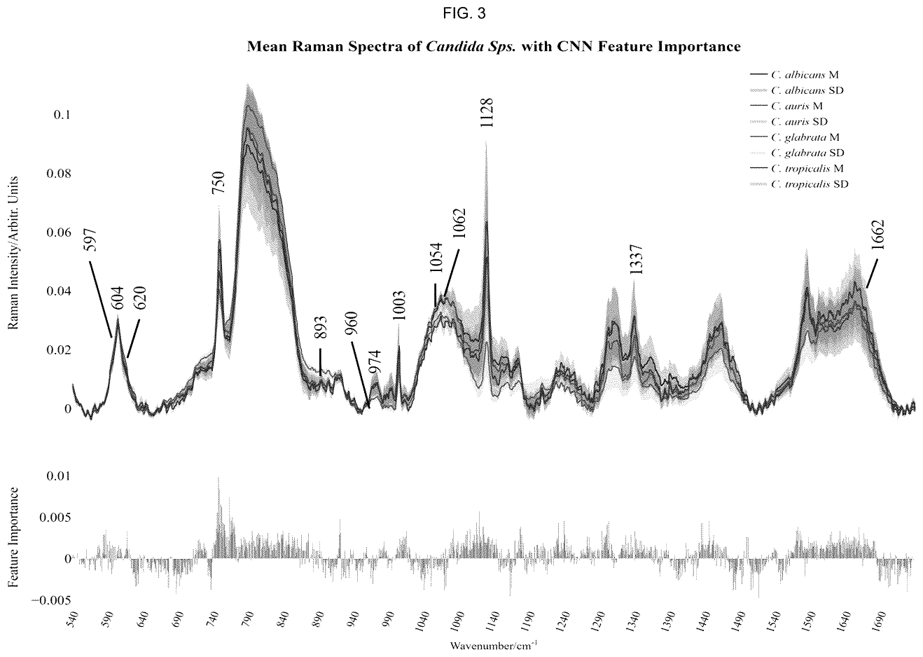

Significant Raman features. The Raman spectra of Candida species (FIG. 3) reveal key biological features alongside overlapping bands attributed to the cuvette material and water. The broad Raman peaks between 750-850 cm⁻¹ and 1010-1270 cm⁻¹ are associated with the fused silica of the cuvette window. The prominent peak at 1641 cm⁻¹ is associated with the water in the suspension and the cells of most biological samples. Despite these background signal contributions, distinct variations in the Raman spectra of the four Candida species are observed. These variations reflect differences in cellular energy states (mitochondrial cytochromes b and c), cell wall composition (β-glucan, chitin, and mannoprotein), and cell membrane components (ergosterol), providing the basis for analysis.

Permutation importance was conducted to ascertain Raman features that most significantly impacted the classification outcomes (FIG. 3). This analysis identified influential features clustered within specific spectral regions instead of discrete peaks. Although previous studies have emphasized specific Raman peaks as crucial for distinguishing Candida species (Dumouilla & Dussap, Bioengineered 2021, 12, 4420; Mikkelsen et al., Food Research International 2010, 43, 2417; Pezzotti et al., International Journal of Molecular Sciences 2022, 23, 11736; Thompson et al., Frontiers in Microbiology 2019, 10), employing a limited number of features resulted in a suboptimal model with inadequate capability to differentiate Candida species. The deep learning model used in this example examined the entire spectrum rather than relying on isolated peaks. This integrative methodology enables the model to discern intricate interactions among various spectral features, thereby enhancing its capability to differentiate between species accurately. This improvement is attributable to the ability of CNNs to capture complex interactions among various spectral features. CNNs achieve this by employing hierarchical feature extraction, where initial layers identify simple features such as individual peaks, and deeper layers recognize more complex patterns, including interactions between peaks. Furthermore, CNNs utilize local connectivity and shared weights, enabling the detection of local patterns and their combinations across the spectrum. Nonlinear activation functions within CNNs also facilitate the modeling of non-linear relationships between spectral features, allowing for a comprehensive understanding of intricate interactions. By learning feature representations directly from the data, CNNs can identify and leverage subtle interactions that traditional methods might overlook (Deneu et al., PLOS Computational Biology 2021, 17, e1008856; Zeng et al., In 2021 IEEE International Performance, Computing, and Communications Conference (IPCCC); IEEE: 2021, p 1). Although many features were important to the CNN model, bar graphs were generated to examine notable differences in the Raman peak intensities at characteristic wavenumbers between Candida species.

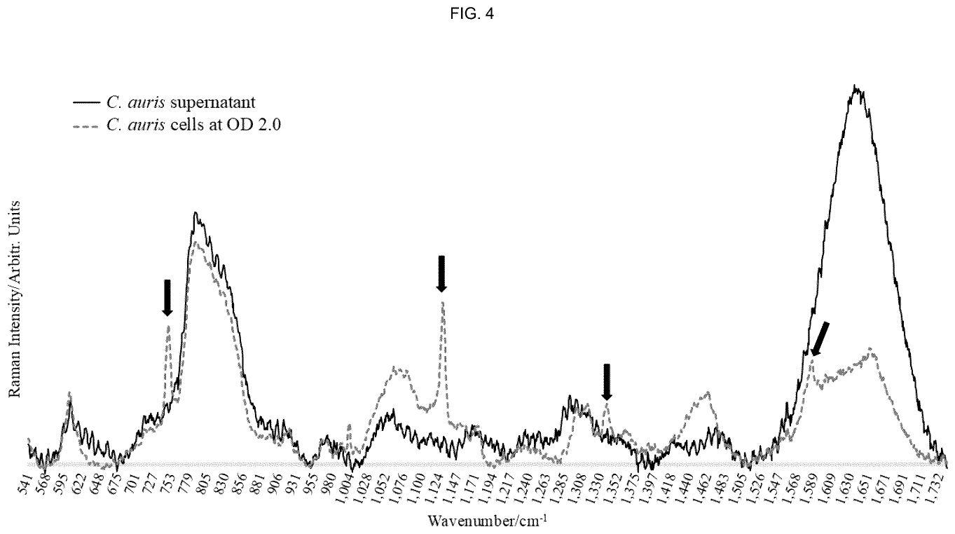

Cellular energy states. Changes in yeast cell physiology can be effectively monitored by assessing the respiratory activity of mitochondria in vivo using resonance Raman spectroscopy, a variant of conventional Raman spectroscopy (Kakita et al., Journal of Biophotonics 2012, 5, 20). This method quantifies the redox state of mitochondrial Cytochrome b and c, pivotal components of the electron transfer system, by excitation at a wavelength of 532 nm to induce resonance (Adar, Spectroscopy 2013, 28). The excitation wavelength for CPGB-RS, set at 532 nm, induces resonance at 750, 1129, 1340, and 1585 cm⁻¹ peaks associated with Cytochromes b and c, facilitating the observation of Candida's energy states in vivo (FIG. 4). The oxidized forms of cytochromes contain ferric heme, whereas the reduced forms contain ferrous heme. Heme proteins exhibit strong coloration, and when the wavelength of the excitation source aligns with the molecule's electronic transition, the intensity of the Raman-active vibration is enhanced (Kakita et al., Journal of Biophotonics 2012, 5, 20; Adar, Spectroscopy 2013, 28; Brown et al., Biophysica Acta (BBA)-Bioenergetics 2008, 1777, 877). However, it is important to note that the intensity does not increase linearly with concentration. Additionally, the molecule's color can result in self-absorption, diminishing the signal at higher concentrations (Adar, Spectroscopy 2013, 28).

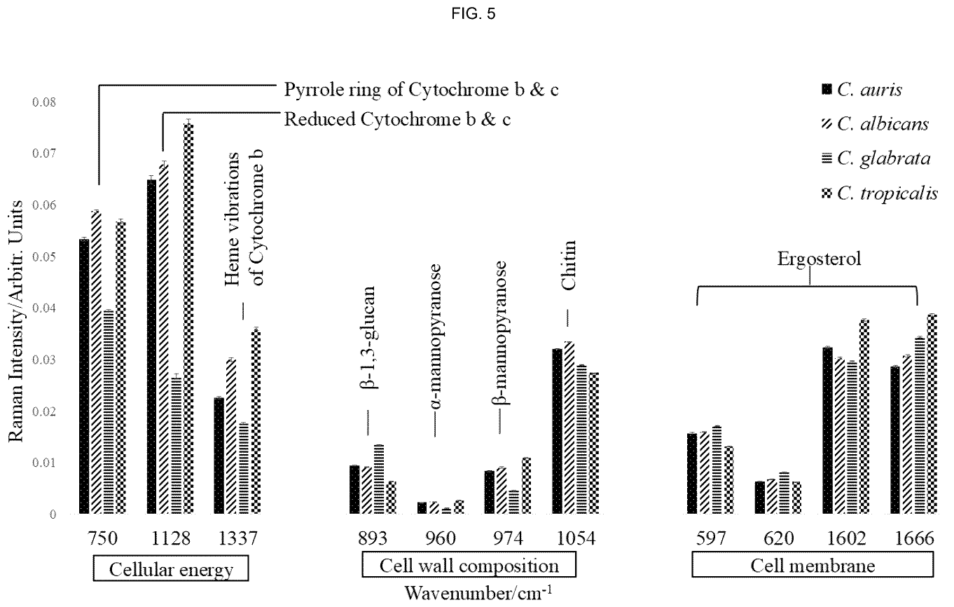

While the Raman spectra of oxidized and reduced states of Cytochromes b and c have common bands, distinctive peaks that serve as unique identifiers exist, unique to Cytochrome c, is a characteristic peak at 604 cm⁻¹ found in reduced state, and a peak at 1313 cm⁻¹ present in both the oxidized and reduced forms. These bands are absent in the spectra of both reduced and oxidized Cytochrome b, providing a clear differentiation between the Cytochromes (Kakita et al., Journal of Biophotonics 2012, 5, 20). Similarly, the spectra of Cytochrome b have peaks at 1303 and 1337 cm⁻¹, found in both the oxidized and reduced forms, that are absent in spectra of Cytochrome c. In the oxidized state, both Cytochromes b and c exhibit a peak at 1638 cm⁻¹ that is absent in the reduced states (Kakita et al., Journal of Biophotonics 2012, 5, 20). Two important peaks at 750 and 1128 cm⁻¹, which signify breathing vibrations of the heme pyrrole ring in Cytochromes b and c, indicative of reduced forms, have been used to represent the fraction of Cytochrome c in the mitochondria of Candida cells (Zhu et al., Spectrochimica Acta Part A: Molecular and Biomolecular Spectroscopy 2011, 78, 1187; Chen et al., Q Analyst 2020, 145, 3922; Welchen & Gonzalez, Physiologia Plantarum 2016, 157, 310; Alvarez-Paggi et al., Chemical reviews 2017, 117, 13382; Wu et al., Free Radical Biology and Medicine 2022, 184, 1). A higher peak intensity at 750 and 1128 cm⁻¹ implies more energetically active Candida cells, whereas a weaker peak intensity suggests dormant cells (Pezzotti et al., International Journal of Molecular Sciences 2022, 23, 11736). Furthermore, Candida species that form large biofilms tend to have a weaker 750 cm⁻¹ band intensity (Pezzotti et al., International Journal of Molecular Sciences 2022, 23, 11736). While spectral bands centered around 1128 and 1337 cm⁻¹ were found significant to the CNN classification model, a bar graph was generated to investigate Raman peaks at discrete wavenumbers. The graph demonstrates differences in peak intensities between Candida species at 750, 1128, and 1337 cm⁻¹ (FIG. 5).

To ensure that the Raman bands corresponding to Cytochromes b and c are in vivo, spectra of the sample supernatant were acquired. Raman peaks indicative of Cytochromes b and c are absent in the supernatant (FIG. 4). Despite the importance of these markers, relying solely on them for classification is not advisable, as the cell's energy states can vary depending on environmental conditions. Furthermore, the cell's energy states can fluctuate based on environmental conditions and possible photobleaching (Okotrub & Surovtsev, Journal of Photochemistry and Photobiology B: Biology 2014, 141, 269).

Cell wall composition. The principal constituents of the Candida cell wall are β-glucans, chitin, and mannoproteins, which are arranged in a two-layered configuration. The inner layer primarily includes chitin, an unbranched polymer of β-1,4-N-acetyl-d-glucosamine, intertwined with β-1,3-glucan, a glucose polymer with β-1,6 linkages that connect the inner and outer layers. This structural arrangement collectively forms a scaffold resembling a basket that imparts structural rigidity to the cell (Calderone & Braun, Microbiological reviews 1991, 55, 1; Cassone, Current topics in medical mycology 1989, 248; Shepherd et al., Annual review of microbiology 1985, 39, 579; Shepherd, Critical Reviews in Microbiology 1987, 15, 7). Mannoproteins, composed of mannose units covalently attached to proteins, form the outer layer and contribute to immune evasion and virulence (Chaffin et al., Microbiol Mol Biol Rev 1998, 62, 130; Gow et al., Microbiology spectrum 2017, 5, 10.1128). Although some fungi incorporate α-glucans into their cell wall, Candida species notably lack this component (Gow et al., Microbiology spectrum 2017, 5, 10.1128; Damveld et al., Fungal Genetics and Biology 2005, 42, 165; Klis et al., Medical mycology 2001, 39, 1; Yoshimi et al., Journal of fungi 2017, 3, 63).

Candida species intricately regulate their cell wall architecture in response to environmental cues, thereby enabling persistence, resistance to antifungal drugs, and evasion of immune surveillance (Gow et al., Microbiology spectrum 2017, 5, 10.1128). Cell wall morphology and composition variations contribute to species-specific characteristics (Pezzotti et al., International Journal of Molecular Sciences 2022, 23, 11736; Seneviratne et al., Oral Diseases 2008, 14, 582). These distinct compositions serve as targets for differentiation and identification of Candida, even between clades of the same species, through Raman spectroscopy (Pezzotti et al., International Journal of Molecular Sciences 2022, 23, 11736; Pezzotti et al., Int J Mol Sci 2022, 23). For CNN classification, spectral bands centered around 1003, 1174, and 1460 cm⁻¹ are impactful to the deep learning algorithm and are attributed to vibrational modes of proteins (Phe at 1003 and 1174 cm⁻¹) and lipids (CH₂/CH₃ deformations at 1460 cm⁻¹).

To further investigate differences in cell wall composition between species, a literature survey was conducted. Compared with C. albicans and C. tropicalis, C. auris is reported to demonstrate elevated chitin levels in its cell wall (Navarro-Arias et al., Infection and drug resistance 2019, 783). Chitin backbone structure produces a triplet of Raman peaks at 1054, 1107, and 1147 cm⁻¹, along with a low frequency peak at 645 cm⁻¹ (De Gussem et al., Spectrochimica Acta Part A: Molecular and Biomolecular Spectroscopy 2005, 61, 2896). Additionally, skeletal stretching (C—C and C—O) among polysaccharides generates a Raman peak at 1123 cm⁻¹ (Pezzotti et al., International Journal of Molecular Sciences 2022, 23, 11736). Chitin substructures, including Amide I, II, and Ill groups, yield Raman peaks between 1600-1700 cm⁻¹, 1400-1500 cm⁻¹, and 1300 cm⁻¹, respectively (Pezzotti et al., International Journal of Molecular Sciences 2022, 23, 11736). For α-chitin, amide I manifests a doublet Raman peak at 1644 and 1660 cm⁻¹, representing two different types of hydrogen bonds (Focher et al., Carbohydrate Polymers 1992, 17, 97; Zhang et al., The journal of physical chemistry B 2012, 116, 4584; Minke & Blackwell, J Journal of molecular biology 1978, 120, 167). A bar graph reveals increased peak intensity at 1054 cm⁻¹ in C. auris, indicating elevated chitin levels compared to other Candida species (FIG. 5).

Among C. glabrata, C. albicans, and C. tropicalis, C. glabrata is reported to exhibit the highest β-glucan levels in its cell walls, followed by C. albicans and C. tropicalis (Thompson et al., Frontiers in Microbiology 2019, 10). Raman peaks corresponding to β-1,3-glucan arising from C—O—C glycosidic linkage vibrations are found at 893 cm⁻¹ (Mikkelsen et al., Food Research International 2010, 43, 2417). A bar graph demonstrates higher levels of β-glucan in C. glabrata, with C. albicans and C. tropicalis following in succession (FIG. 5). Conversely, the mannoprotein levels in C. glabrata are reported to be notably lower than those in C. tropicalis and C. albicans (Thompson et al., Frontiers in Microbiology 2019, 10). Mannoproteins, specifically α- and β-mannopyranose forms, manifest Raman bands at 960 and 974 cm⁻¹, respectively (Dumouilla & Dussap, Bioengineered 2021, 12, 4420). The bar graph indicates decreased levels of β mannopyranose in C. glabrata (FIG. 5).

Cell membrane components. Ergosterol, a primary sterol component in the Candida cell membrane, plays a pivotal role in the functionality of the cell membranes, particularly in facilitating the transport process and the enzymatic activities of membrane-bound enzymes (Breivik & Owades, Journal of Agricultural and Food Chemistry 1957, 5, 360; Demel & De Kruyff, Biochimica et Biophysica Acta (BBA)-Reviews on Biomembranes 1976, 457, 109). Most antifungal drugs used to treat Candida infections target the ergosterol biosynthetic pathway (Sanglard et al., Antimicrobial agents and chemotherapy 2003, 47, 2404). Disruption of ergosterol biosynthesis results in compromised cell membrane permeability (Bard et al., Journal of Bacteriology 1978, 135, 1146). Candida species resistant to antifungal agents such as Azole have been reported to have higher levels of ergosterol in their membranes (Zhang et al., Analytical Chemistry 2023). In Raman spectroscopy, ergosterol exhibits characteristic bands at 597 and 620 cm⁻¹, reflecting in-plane ring deformation modes associated with membrane sterols, and at 1602 cm⁻¹ and 1666 cm⁻¹, corresponding to C═C stretching within rings and in the acyl chains, respectively (Pezzotti et al., International Journal of Molecular Sciences 2022, 23, 11736; Živanović et al., J Analytical Chemistry 2018, 90, 8154). In the currently described findings, ergosterol peaks at 597, 620, 1602, and 1666 cm⁻¹ have overlapping contributions from reduced Cytochrome c at 604 cm⁻¹ and Amide I between 1600-1700 cm⁻¹. Despite the overlap, permutation importance analysis shows Raman features centered around 1666 cm⁻¹ to be significant to the CNN model (FIGS. 3 and 4). In future studies, analyzing changes in the intensity of Raman peaks for ergosterol in response to antifungal agents will facilitate the investigation of the efficacy of agents targeting the cell membrane.

Conclusions. The CPGB-RS is a portable table-top Raman spectroscopy-based diagnostic platform that allows rapid evaluation of Candida auris and other Candida species affecting human health. This example demonstrates the use of the system combined with a CNN deep learning model to accurately distinguish between C. auris, C. albicans, C. glabrata, and C. tropicalis, achieving a high accuracy of 96%, weighted sensitivity of 96%, and weighted specificity of 99%. The CPGB-RS platform offers several advantages over traditional diagnostic modalities, such as culture-based methods, MALDI-TOF MS, and conventional PCR, by enabling rapid, reagentless screening without requiring complex sample preparation or operation by highly trained personnel. The system can provide software-generated results within two minutes, making it particularly suitable for use in resource-limited settings or smaller clinical laboratories that may not have access to advanced diagnostic equipment. Moreover, with the use of the Raman spectral database and employing adaptive algorithms, this technology can detect new and emerging strains, thereby serving as a versatile diagnostic tool.

### Example Clauses

1. A system for identifying fungal pathogens, including: a Raman spectrometer with a counter-propagating Gaussian beam; a sample chamber; and a processor configured with a machine learning algorithm configured to analyze Raman spectral data and identify fungal species.

2. The system of clause 1 or 2, wherein the counter-propagating Gaussian beam enhances signal intensity.

3. The system of any of clauses 1-3, wherein the sample chamber is optimized for low-concentration sample analysis.

4. The system of any of clauses 1-4, wherein the machine learning algorithm is a convolutional neural network (CNN).

5. The system of clause 4, wherein the CNN includes a plurality of layers and wherein a layer, of the plurality of layers, includes a filter associated with one or more parameters.

6. The system of clause 4 or 5, wherein the CNN is trained based on training data including example input images and corresponding example outputs.

7. The system of any of clauses 1-6, wherein the fungal species include Candida.

8. The system of clause 7, wherein the Candida includes Candida. auris, Candida albicans, Candida glabrata, or Candida tropicalis.

9. The system of any of clauses 1-8, wherein the Raman spectral data includes features indicative of fungal species.

10. The system of clause 9, wherein the features indicative of fungal species include variations in cell wall composition, cell membrane components, and cellular energy states.

11. The system of any of clauses 1-10, further including a user interface for automated result display.

12. The system of any of clauses 1-11, further including a user interface for clinical decision support.

13. The system of clause 1, wherein the system generates, based on the identified fungal species, a report.

14. The system of clause 13, wherein the report recommends a therapy.

15. The system of clause 14, wherein the recommended therapy includes a dosage of one or more therapeutic agents predicted to treat a condition of a subject associated with the identified fungal species.

16. The system of clause 13 or 14, wherein the system transmits the report to an external device.

17. The system of clause 16, wherein the external device is associated with a subject and/or a healthcare provider.

18. The system of clause 16 or 17, wherein the system transmits the data over one or more communication networks.

19. The system of any of clauses 16-18, wherein the system transmits the data over a peer-to-peer connection.

20. The system of any of clauses 1-19, wherein the machine learning algorithm utilizes additional spectral processing techniques.

21. The system of clause 20, wherein the additional spectral processing techniques include adaptive learning for new species identification.

22. The system of clause 20 or 21, wherein the additional spectral processing techniques include real-time anomaly detection.

23. The system of any of clauses 1-22, wherein the system is portable.

24. The system of any of clauses 1-23, wherein the Raman spectrometer with a counter-propagating Gaussian beam is equipped with a 532 nm laser.

25. The system of clause 24, wherein the system includes a cuvette that concentrates particles at the 532 nm laser's focal point.

26. The system of any of clauses 1-25, wherein the Raman spectrometer includes a high-resolution spectrometer that separates wavelengths onto a cooled CCD detector for data acquisition.

27. The system of any of clauses 1-26, wherein the Raman spectrometer records Raman spectra over a range of 285-1925 cm⁻¹.

28. The system of any of clauses 1-27, wherein the system performs preprocessing.

29. The system of clause 28, wherein the preprocessing includes one or more of baseline subtraction, spectral range optimization (540-1740 cm⁻¹), or normalization to improve signal-to-noise ratios.

30. The system of any of clauses 1-29, wherein the system removes outlier spectra.

31. The system of any of clauses 1-30, wherein the identifying of the fungal species occurs within two minutes of receiving a sample within the system.

32. Use of the system of any of clauses 1-31, for evaluating antifungal resistance by monitoring spectral changes in response to therapeutic agents.

33. Use of the system of any of clauses 1-31 wherein spectral biomarkers indicative of antimicrobial resistance or susceptibility are analyzed to determine minimum inhibitory concentrations.

34. The use of clause 33, wherein minimum inhibitory concentrations are determined within 30 minutes.

35. A method for diagnosing fungal infections, including: preparing a biological sample and acquiring Raman spectral data using a counter-propagating Gaussian beam Raman spectroscopy (CPGB-RS) system; processing the Raman spectral data to enhance signal quality; analyzing the processed Raman spectral data using a machine learning algorithm to identify fungal species; and generating results diagnosing the fungal infections.

36. The method of clause 35, wherein the generating occurs within two minutes of the analyzing.

37. The method of clause 35 or 36, wherein the machine learning algorithm is a convolutional neural network (CNN).

38. The method of clause 37, wherein the CNN includes a plurality of layers and wherein a layer, of the plurality of layers, includes a filter associated with one or more parameters.

39. The method of clause 37 or 38, wherein the CNN is trained based on training data including example input images and corresponding example outputs.

40. The method of any of clauses 35-39 wherein the biological sample include Candida.

41. The method of clause 40, wherein the Candida includes Candida. auris, Candida albicans, Candida glabrata, or Candida tropicalis.

42. The method of any of clauses 35-41, wherein the Raman spectral data includes features indicative of a fungal species.

43. The method of clause 42, wherein the features indicative of the fungal species include variations in cell wall composition, cell membrane components, and cellular energy states.

44. The method of any of clauses 35-43, wherein the processing Raman spectral data includes one or more of baseline subtraction, spectral range optimization (540-1740 cm⁻¹), normalization to improve signal-to-noise ratios, or outlier removal.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to detect and differentiate fungal species as described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

1. A system for identifying fungal pathogens, comprising:
a Raman spectrometer with a counter-propagating Gaussian beam;
a sample chamber; and
a processor coupled to the Raman spectrometer and the sample chamber configured to perform, via a convolutional neural network (CNN), operations comprising:
receiving Raman spectral data captured by the Raman spectrometer, the Raman spectral data comprising features indicative of fungal species; and
identifying fungal species.

2-6. (canceled)

7. The system of claim 1, wherein the fungal species comprise Candida.

8. The system of claim 7, wherein the Candida comprises Candida auris, Candida albicans, Candida glabrata, or Candida tropicalis.

9. (canceled)

10. The system of claim 1, wherein the features indicative of fungal species comprise species variations in at least one of cell wall composition, cell membrane components, or cellular energy states.

11-14. (canceled)

15. The system of claim 1, wherein the system generates a recommended therapy comprising a dosage of one or more therapeutic agents predicted to treat a condition of a subject associated with the identified fungal species.

16-20. (canceled)

21. The system of claim 1, wherein the processor is further configured to perform adaptive learning for new species identification by training the CNN at periodic intervals using a dataset comprising an emerging pathogen classification library.

22. The system of claim 1, wherein the processor is further configured to perform real-time anomaly detection by:
identifying at least one feature in the Raman spectral data that deviates from a threshold; and
determining a presence of an anomaly based on the feature.

23. (canceled)

24. The system of claim 1, wherein the Raman spectrometer with a counter-propagating Gaussian beam is equipped with a laser having an excitation wavelength of 532 nm.

25. The system of claim 24, wherein the system comprises a cuvette that concentrates particles at a focal point of the laser.

26. The system of claim 1, wherein the Raman spectrometer comprises a high-resolution spectrometer that separates wavelengths onto a cooled CCD detector for data acquisition.

27. The system of claim 1, wherein the Raman spectrometer records Raman spectra over a range of 285-1925 cm⁻¹.

28-34. (canceled)

35. A method for diagnosing fungal infections, comprising:
preparing a biological sample;
acquiring, using a counter-propagating Gaussian beam Raman spectroscopy (CPGB-RS) system, Raman spectral data comprising features indicative of fungal species;
preprocessing the Raman spectral data to enhance signal quality; and
analyzing the preprocessed Raman spectral data using a convolutional neural network (CNN) to:
identify fungal species; and
generate results diagnosing the fungal infections.

36-39. (canceled)

40. The method of claim 35, wherein the biological sample comprises Candida.

41. The method of claim 40, wherein the Candida comprises Candida auris, Candida albicans, Candida glabrata, or Candida tropicalis.

42. (canceled)

43. The method of claim 35, wherein the features indicative of the fungal species comprise species variations in at least one of cell wall composition, cell membrane components, or cellular energy states.

44. (canceled)

45. The system of claim 1, wherein the CNN is trained using a Raman spectral database comprising:
one or more features indicative of species variations in at least one of cell wall composition, cell membrane components, or cellular energy states; or
one or more spectral biomarkers indicative of antimicrobial resistance or susceptibility.

46. The method of claim 35, wherein preprocessing Raman spectral data comprises truncating the Raman spectral data across a specific spectral range of 540-1740 cm⁻¹ to generate a truncated spectral range.

47. The method of claim 46, wherein preprocessing Raman spectral data further comprises:
identifying local minimum points within the truncated spectral range;
fitting a background signal to the local minimum points using a penalized least squares operation constrained by a weighted vector;
generating baseline-corrected data by subtracting the background signal from the truncated spectral range;
generating normalized spectral data from the baseline-corrected data using a unit vector; and
filtering the normalized spectral data to remove spectral outliers.

48. The method of claim 35, wherein the Raman spectral data comprises spectral biomarkers indicative of antimicrobial resistance or susceptibility, and wherein analyzing the preprocessed Raman spectral data further comprises determining minimum antimicrobial agent sufficient to inhibit fungal infections.

49. The method of claim 35, wherein analyzing the preprocessed Raman spectral data further comprises evaluating antifungal resistance by monitoring spectral changes in response to one or more therapeutic agents.
